(19) United States
(12) Patent Application Publication
(10) Pub. No.: US 2012/0083693 A1
(43) Pub. Date: Apr. 5, 2012

(54) HANDHELD ULTRASONIC DIAGNOSTIC APPARATUS WITH RACK STAND

(75) Inventor: Sung In CHO, Seoul (KR)

(73) Assignee: SAMSUNG MEDISON CO., LTD.

(21) Appl. No.: 13/248,719

(22) Filed: Sep. 29, 2011

(30) Foreign Application Priority Data
Sep. 30, 2010 (KR) 10-2010-0095392

(51) Int. Cl. A61B 8/00

(52) U.S. Cl. 600/437

(57) ABSTRACT

The present disclosure relates to a handheld ultrasonic diagnostic apparatus that includes a height adjustable rack stand. The apparatus includes a portable main body, and a rack stand that supports the main body such that the main body can be held at a preset height. The handheld ultrasonic diagnostic apparatus can be rapidly installed using the rack stand, thereby enabling a user to rapidly address an emergency situation using the apparatus.

### TECHNICAL FIELD

The present disclosure relates to an ultrasonic diagnostic apparatus and, more particularly, to a handheld ultrasonic diagnostic apparatus that includes a height-adjustable rack stand.

### BACKGROUND

Ultrasonic diagnostic apparatuses are applied a variety of fields. For example, ultrasonic diagnostic apparatuses are extensively used in a medical field due to non-invasive and non-destructive characteristics to human bodies. Recently, high performance ultrasonic diagnostic apparatuses have been used to provide two-dimensional or three-dimensional images of internal organs of the human bodies.

Such an ultrasonic diagnostic apparatus is generally large and heavy, so even a small ultrasonic diagnostic apparatus has a heavy weight of about 10 kg or more. The large and heavy ultrasound diagnostic apparatus is difficult to carry and be handheld. To overcome such problems of the existing ultrasonic diagnostic apparatus, a handheld ultrasonic diagnostic apparatus has been developed.

It should be noted that the above description is provided for understanding of the background art and is not a description of a well-known conventional technique to which the present disclosure pertains.

Generally, a handheld ultrasonic diagnostic apparatus does not include a suitable rack stand and is used on a desk or mattress. As a result, when there is no desk or mattress to support the apparatus or the apparatus is in an environment unfavorable for use at a desk or mattress, it is difficult for a user to rapidly address an emergency using the handheld ultrasonic diagnostic apparatus.

Furthermore, since the handheld ultrasonic diagnostic apparatus is not easy-to-carry, a separate bag must be provided to hold the handheld ultrasonic diagnostic apparatus.

Hence, a need exists for an improved handheld ultrasonic diagnostic apparatus that overcomes such problems.

### SUMMARY

To improve the art and address the above and other needs, the present disclosure encompasses a handheld diagnostic medical apparatus including a rack stands that is easily portable and that easily handles emergency situations.

An aspect of the disclosure relates to a handheld ultrasonic diagnostic apparatus including a main body; and a rack stand that supports the main body such that the main body can be held at a preset height.

The rack stand may be connected to the main body to rotate about a point on the main body. Here, the rack stand may be stretchable from the hinge point of the main body.

The main body may be provided with a guide on a surface thereof and the rack stand may include a plurality of rack members. Here, each of the rack members may be disposed at one side thereof in the guide to move along the guide and is in contact with the other side thereof with a floor to support the main body. Specifically, one of the rack members may be disposed at one side thereof in the guide and may be in contact with the other side thereof with the floor, and the other rack member may be disposed at one side thereof in the guide and may be in contact with the other side thereof with the floor. The one rack member and the other rack member are coupled to each other to rotate about a hinge point between the rack members to support the main body at different heights according to the degree of rotation of the rack members.

The main body may be provided with a mounting portion and the rack stand may be stretchable from the mounting portion to a side opposite the main body. The rack stand may include rack members disposed separately from each other; and crossing members disposed between the rack members to raise or lower the rack members. The crossing members may be disposed to cross each other at a hinge point between the crossing members and may rotate about the hinge point to raise or lower the rack members.

The rack stand may be disposed to surround the main body. The rack stand may include a first rack member provided to the main body, a second rack member rotatably coupled to the first rack member, and a third rack member rotatably coupled to the second rack member and protecting the main body.

The rack stand may be detachably coupled to the main body.

### BRIEF DESCRIPTION OF THE DRAWINGS

The figures depict one or more implementations in accordance with the present disclosure, by way of example only, not by way of limitation. In the figures, like reference numerals refer to the same or similar elements.

FIG. 1 is a top perspective view of a handheld ultrasonic diagnostic apparatus with a rack stand in accordance with the disclosure;

FIG. 2 is a bottom perspective view of a handheld ultrasonic diagnostic apparatus with a rack stand in accordance with a first embodiment of the disclosure;

FIG. 3 illustrates an operation of the rack stand of FIG. 2;

FIG. 4 is a bottom perspective view of a handheld ultrasonic diagnostic apparatus with a rack stand in accordance with a second embodiment of the disclosure;

FIG. 5 illustrates an operation of the rack stand of FIG. 4;

FIG. 6 is a bottom perspective view of a handheld ultrasonic diagnostic apparatus with a rack stand in accordance with a third embodiment of the disclosure;

FIG. 7 illustrates an operation of the rack stand of FIG. 6;

FIG. 8 is a side view of a handheld ultrasonic diagnostic apparatus with a rack stand in accordance with a fourth embodiment of the disclosure; and

FIG. 9 illustrates operation of the rack stand of FIG. 8.

### DETAILED DESCRIPTION

Exemplary embodiments of the disclosure will now be described in detail with reference to the accompanying drawings. It should be noted that the drawings are not in a precise scale and may be exaggerated in thickness of lines or size of components for descriptive convenience and clarity only. Furthermore, terms used herein are defined by taking functions of the disclosure into account and can be changed according to the custom or intention of users or operators. Therefore, the terms should be defined according to the overall disclosures set forth herein.

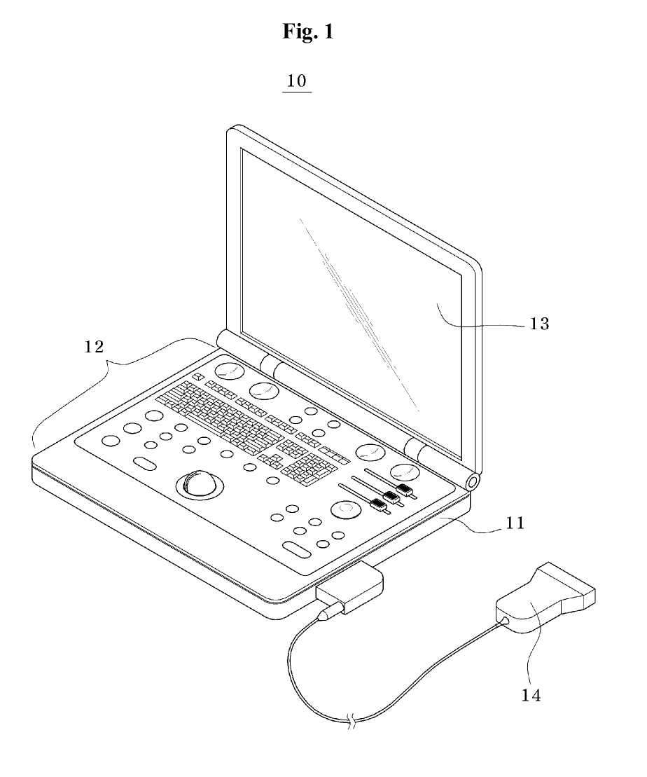

FIG. 1 is a top perspective view of a handheld ultrasonic diagnostic apparatus with a rack stand in accordance with the disclosure.

Referring to FIG. 1, the handheld ultrasonic diagnostic apparatus 10 includes a portable main body 11, a control panel 12, a display unit 13, and a probe 14.

The main body 11 defines an external appearance of the apparatus 10 and is powered by a battery or other external power sources. The main body 11 is connected to the probe 14 that emits ultrasound waves towards an object and converts the ultrasound waves reflected from the object into electrical signals. The main body 11 is provided with an electric circuit which processes analog and digital signals for ultrasonic diagnosis.

The control panel 12 is disposed on the main body 11 and includes a plurality of input keys for performing functions of obtaining or adjusting diagnostic images, controlling menus, measurement and annotation, etc.

The display unit 13 displays data and images sent from the main body 11, which processes the data and images.

The probe 14 includes at least one transducer (not shown). The transducer sends ultrasound waves to an object and receives the ultrasound waves reflected therefrom. The handheld ultrasonic diagnostic apparatus 10 is small and light, so easy to carry.

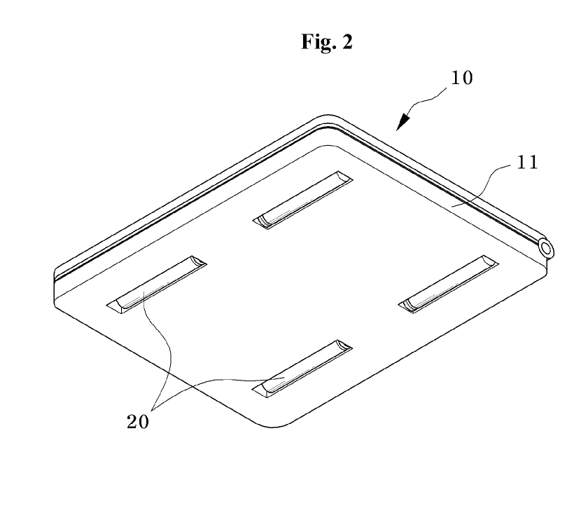

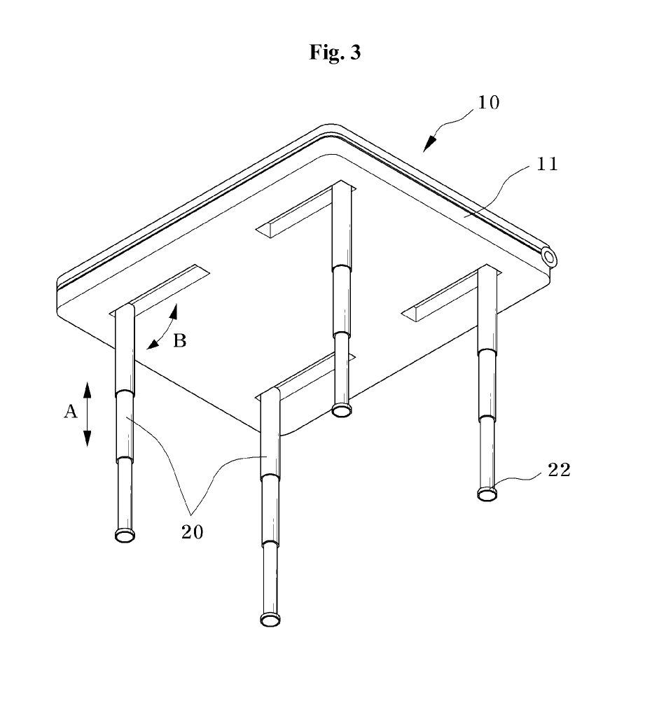

FIG. 2 is a bottom perspective view of a handheld ultrasonic diagnostic apparatus with a rack stand in accordance with a first embodiment of the disclosure and FIG. 3 illustrates an operation of the rack stand of FIG. 2.

Referring to FIGS. 2 and 3, the handheld ultrasonic diagnostic apparatus 10 according to this embodiment includes a rack stand 20 which supports the main body 11 such that the main body 11 can be held at a preset height. The configuration of the rack stand 20 may be realized in various ways. For example, the rack stand 20 may be secured to the main body 11. Alternatively, the rack stand 20 may be detachably coupled to the main body 11. Since configuration of detachably coupling the rack stand 20 to the main body 11 is well known to a person having ordinary knowledge in the art, a detailed description thereof will be omitted herein.

The rack stand 20 is provided to the main body 11 to rotate about a point (not shown) on the main body 11. Namely, when a user uses the rack stand, the rack stand 20 may rotate in a direction of Arrow “B” as needed, for example, in case that there is no desk or mattress to support the main body 11. Further, as shown in FIG. 3, the rack stand 20 may stretch in a direction of Arrow “A” from the point of the main body 11 to allow a user to use the diagnostic apparatus 10 at a desired height. The rack stand 20 may be provided at one end thereof with a support plate 22, which prevents the rack stand 20 from sliding across a floor.

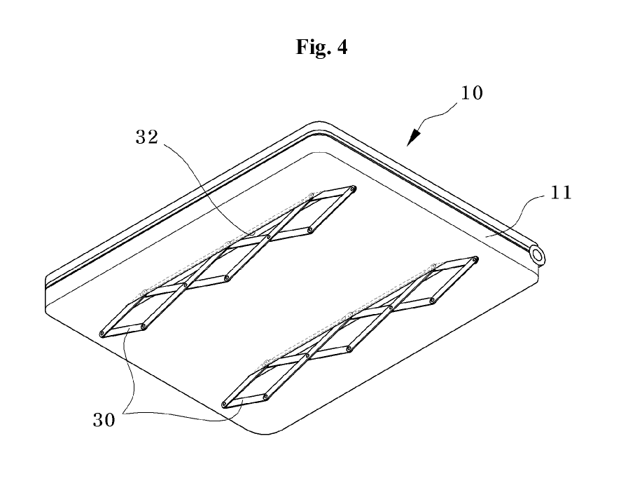

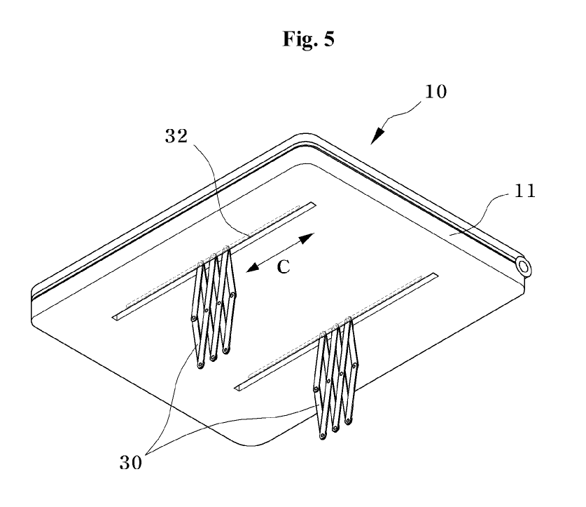

FIG. 4 is a bottom perspective view of a handheld ultrasonic diagnostic apparatus with a rack stand in accordance with a second embodiment of the disclosure and FIG. 5 illustrates an operation of the rack stand of FIG. 4.

Referring to FIGS. 4 and 5, the handheld ultrasonic diagnostic apparatus 10 according to this embodiment includes a rack stand 30 which supports the main body 11 such that the main body 11 can be held at a preset height.

In this embodiment, the main body 11 is provided with a guide 32 on a bottom surface of the main body 11. The guide 32 has an elongated groove shape in which the rack stand 20 can move in a direction of Arrow “C”. The rack stand 20 includes rack members, each of which is disposed at one side thereof in the guide 32 and is movable along the guide 32 to contact with the other side thereof with a floor to support the main body 11. Specifically, as shown in FIG. 4, one of the rack members may be disposed at one side thereof in the guide 32 and is movable to contact with the other side thereof with the floor, and the other rack member may be disposed at one side thereof in the guide 32 and is movable to contact with the other side thereof with the floor. Here, the one rack member and the other rack member are coupled to each other to rotate about a hinge point (not shown) between the rack members to support the main body at different heights according to the degree of rotation of the rack members. In this embodiment, the rack members cross each other to form an X-shape and are rotatably hinge-coupled at the hinge point where the rack members cross each other. Further, the guide 32 may be provided with a securing groove for securing the rack stand 30 at a particular location so as to allow the rack stand 30 to support the main body 11 at a desired height. Since this configuration is well known to a person having ordinary knowledge in the art, a detailed description thereof will be omitted herein.

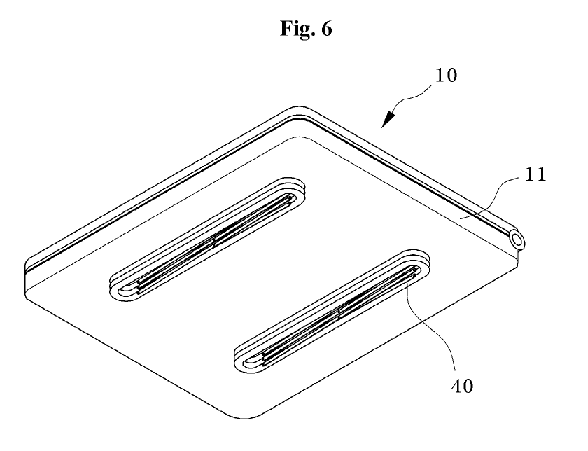

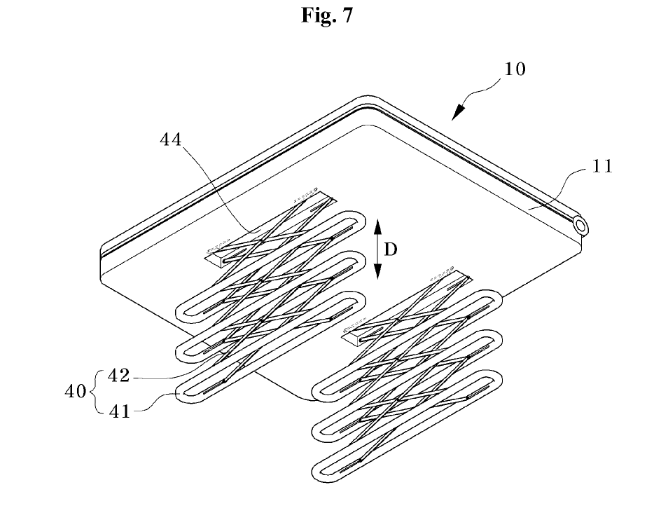

FIG. 6 is a bottom perspective view of a handheld ultrasonic diagnostic apparatus with a rack stand in accordance with a third embodiment of the disclosure and FIG. 7 illustrates an operation of the rack stand of FIG. 6.

Referring to FIGS. 6 and 7, the handheld ultrasonic diagnostic apparatus 10 according to this embodiment includes a rack stand 40 that supports the main body 11 such that the main body 11 can be held at a preset height.

In this embodiment, the main body 11 is provided with a mounting portion 44 and the rack stand 40 is stretchable from the mounting portion 44 to a side opposite to the main body. As shown in FIG. 7, the rack stand 40 includes rack members 41 disposed separately from each other, and crossing members 42 disposed between the rack members 41 to raise or lower the rack members 41 in a direction of Arrow “D”. The crossing members 42 may be disposed to cross each other in an X shape at a hinge point (not shown) where the crossing members are hinge-coupled to each other, and may rotate about the hinge point to raise or lower the rack members 41.

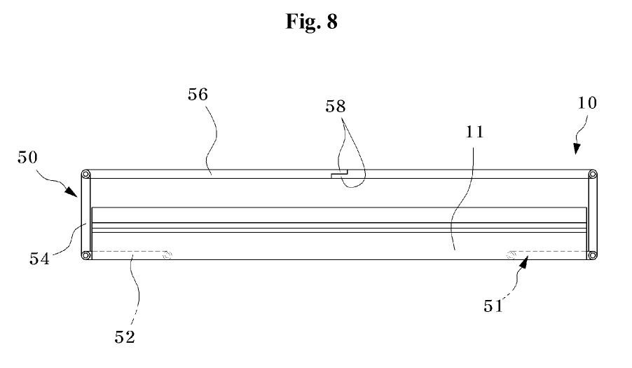

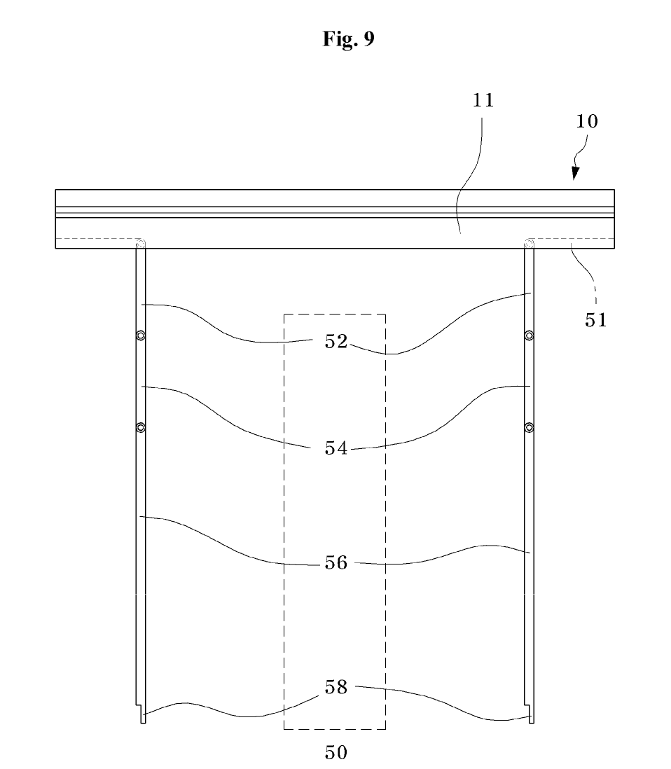

FIG. 8 is a side view of a handheld ultrasonic diagnostic apparatus with a rack stand in accordance with a fourth embodiment of the disclosure and FIG. 9 illustrates an operation of the rack stand of FIG. 8.

Referring to FIGS. 8 and 9, the handheld ultrasonic diagnostic apparatus 10 according to this embodiment includes a rack stand 50 which supports the main body 11 such that the main body 11 can be held at a preset height.

In this embodiment, the rack stand 50 is disposed to surround top, side and bottom surfaces of the main body 11. The rack stand 50 includes a first rack member 52 provided on the bottom surface of the main body 11, a second rack member 54 rotatably coupled to the first rack member 52, and a third rack member 56 rotatably coupled to the second rack member 54 and protecting the main body 11. The first, second and third rack members 52, 54, 56 are provided along the bottom, side and top surfaces of the main body 11 to surround the main body 11. Specifically, referring to FIG. 8, the first, second and third rack members 52, 54, 56 disposed at the bottom, left side and top surfaces of the main body 11 surround the bottom, left side and top surfaces of the main body 11 from the left, and the first, second and third rack members 52, 54, 56 disposed at the bottom, right side and top surfaces of the main body 11 surround the bottom, right side and top surfaces of the main body 11 from the right. Each of the third rack members 56 surrounding the main body 11 from the right and left is provided with a securing portion 58, by which the rack stand 50 can be in a secured state when surrounding the main body 11. The securing portion 58 for connecting and securing the third rack members 56 to each other may be realized in various ways. For example, the securing portions 58 may be realized by the third rack members 56 configured to be mechanically coupled and secured to each other through the securing portions 58. Alternatively, the securing portions 58 may be realized by external structures such as screws, which couple and secure the third rack members 56 to each other. Since the configuration of the securing portion 58 is well known to a person having ordinary knowledge in the art, a detailed description thereof will be omitted herein. The first rack member 52 is connected to a point (not shown) of a mounting groove 51 in the main body 11 to rotate about the point and the second rack member 54 is longer than the height of the main body 11 to allow the third rack member 56 to act as a grip. When the rack stand 50 is used in an emergency or similar situation, the first, second and third rack members 52, 54, 56 rotated to be used as the rack support 50, as shown in FIG. 9.

Each of the rack stands 20, 30, 40, 50 may operate in various ways that fall within the scope of the present disclosure. For example, each of the rack stand 20, 30, 40, 50 may automatically operate by a motor or manually operated by a user.

As such, according to the embodiments, the handheld ultrasonic diagnostic apparatus can be rapidly installed using a rack stand thereof, thereby enabling a user to rapidly address emergency situations using the diagnostic apparatus.

In some embodiments, the rack stand of the handheld ultrasonic diagnostic apparatus may be used as a grip, not only eliminating a separate bag and user fatigue, but also protecting the main body of the apparatus.

While the foregoing has described what are considered to be the best mode and/or other examples, it is understood that various modifications may be made therein and that the subject matter disclosed herein may be implemented in a variety of different ways, and that the disclosure may be applied to numerous applications, only some of which have been described herein. The following claims should be considered to cover all applications, modifications and variations within the true scope of the present disclosure.

What is claimed is:

1. A handheld ultrasonic diagnostic apparatus comprising:
a main body; and
a rack stand disposed on a surface of the main body and supporting the main body to thereby hold the main body at a preset height.

2. The apparatus of claim 1, wherein the rack stand is secured to the surface of the main body to rotate about a point on the main body.

3. The apparatus of claim 2, wherein the rack stand is stretchable from the point of the main body.

4. The apparatus of claim 1, wherein the main body is provided with a guide on the surface of the main body and the rack stand comprises a plurality of rack members, each of the rack members being disposed at one side of the main body in one of the plurality of guides and being movable to along the one of the plurality of the guides to contact at the other side of the main body to support the main body.

5. The apparatus of claim 4, wherein one of the rack members is disposed at one side of the main body in one of the guides and is movable to contact at the other side of the main body, and another of rack members is disposed at one side of the main body in another of the guides and is movable to contact at the other side of the main body, the one rack member and another rack member being coupled to each other to rotate about a hinge point between the rack members to support the main body at different heights according to a degree of rotation of the rack members.

6. The apparatus of claim 1, wherein the main body is provided with a mounting portion and the rack stand is stretchable from the mounting portion in a direction opposite to the main body.

7. The apparatus of claim 6, wherein the rack stand comprises rack members disposed separate from each other, and crossing members disposed between the rack members to raise or lower the rack members.

8. The apparatus of claim 7, wherein the crossing members are disposed to cross each other at a hinge point between the crossing members and rotate about the hinge point to raise or lower the rack members.

9. The apparatus of claim 1, wherein the rack stand is disposed to surround the main body.

10. The apparatus of claim 9, wherein the rack stand comprises a first rack member provided on a bottom surface of the main body, a second rack member rotatably coupled to the first rack member and facing with a side surface of the main body, and a third rack member rotatably coupled to the second rack member and facing a top surface of the main body, and
wherein the first, second and third rack members extend along the bottom, side and top surface respectively to protect the main body when folded and the first, second and third rack members in combination stretch in a direction opposite to the bottom surface of the main body when unfolded.

11. The apparatus of claim 1, wherein the rack stand is detachably coupled to the main body.
